(19) United States
(12) Patent Application Publication
(10) Pub. No.: US 2014/0170098 A1
(43) Pub. Date: Jun. 19, 2014

(54) NAIL VARNISH

(71) Applicant: FABER-CASTELL AG, STEIN (DE)

(72) Inventors: GERHARD LUGERT, NUERNBERG (DE); TATIANA APPEL, OBERASBACH (DE); WOLFGANG KAUL, HEILSBRONN (DE)

(73) Assignee: FABER-CASTELL AG, STEIN (DE)

(21) Appl. No.: 14/107,080

(22) Filed: Dec. 16, 2013

(30) Foreign Application Priority Data
Dec. 14, 2012 (EP) 12197355.6

(51) Int. Cl. A61K 8/58; A61K 8/34; A61K 8/55; A61K 8/25; A61K 8/87; A61Q 3/02; A61K 8/19

(52) U.S. Cl. CPC A61K 8/58; A61Q 3/02; A61K 8/347; A61K 8/19; A61K 8/25; A61K 8/87; A61K 8/55
USPC 424/61

(57) ABSTRACT

The invention relates to a nail varnish having a viscosity of less than 50 mPas (Brookfield LV, DV III Ultra Rheometer, spindle: 40, RPM: 10, 25° C.) and a composition of: 20% to 85% by wt. of an aqueous polymeric dispersion as film former, with a solids fraction of 20% to 60%, 5% to 20% by wt. of a humectant, 1% to 10% by wt. of a colorant formed at least partly of a color pigment, 0.1% to 0.5% by wt. of a phyllosilicate, 0.5% to 5% by wt. of a surfactant, 0% to 5% by wt. of an additive, and remainder of water.

### CROSS-REFERENCE TO RELATED APPLICATION

This application claims the priority, under 35 U.S.C. §119, of European application EP 12 197 355.6, filed Dec. 14, 2012; the prior application is herewith incorporated by reference in its entirety.

### BACKGROUND OF THE INVENTION

### Field of the Invention

The invention relates to a nail varnish which can be applied to a fingernail or toenail by a capillary applicator having a capillary application element and a liquid reservoir. A nail varnish suitable for such application must have a comparatively low viscosity of less than 50 mPas (Brookfield LV, DV III Ultra Rheometer, spindle: 40, RPM: 10, 25° C.) so that it can be applied by a capillary application element, of fibrous nature, for example, interspersed by pores. Nail varnishes of the kind in question, known for example from published, non-prosecuted German patent application DE 199 59 093 A1, contain an aqueous polymer dispersion, and this dispersion, or its polymer fraction, serves primarily to form a film on the nail surface. For coloring there is at least one color pigment present. Also added to the nail varnish is a humectant, which prevents or at least delays the nail varnish drying up in the capillary application element and which also assists film formation by the polymer dispersion. The known nail varnish optionally, lastly, contains additives such as preservatives or care substances.

On prolonged storage of nail varnishes of the present kind, they tend to change color, this being attributable to the sedimentation of color pigments. This is accompanied very generally by a reduction in the pigments dispersed in the aqueous phase, and hence to a reduction in the color intensity. In the case of mixtures of color pigments of different colors, shifts in color may result from the sedimentation of the individual color pigments at different rates.

A further problem is that many dispersants lead to an increase in the viscosity beyond the above-mentioned viscosity limit, and this would be a hindrance to the transport of the nail varnish, based on capillary forces, through the pores of a capillary application element of the applicator. A further problem, finally, is that the dispersant may adversely affect film formation and the quality of the resultant varnish film on the nail surface.

### SUMMARY OF THE INVENTION

It is an object of the invention to propose a nail varnish which provides a remedy to the drawbacks identified above. This object is achieved by a nail varnish according to the invention. The nail varnish of the invention first has a viscosity of less than 50 mPas (Brookfield LV, DV III Ultra Rheometer, spindle: 40, RPM: 10, 25° C.), which is a mandatory prerequisite for the varnish to be capable of application by an applicator having a capillary application element. The varnish is composed of 20% to 85% of an aqueous dispersion of an aliphatic urethane-acrylate hybrid polymer as film former, 5% to 20% of a humectant, 1% to 10% of a colorant formed at least partly of color pigments, 0.1% to 0.5% of a phyllosilicate, 0% to 5%, preferably 0.5% to 3%, of an additive, and 0.5% to 5%, preferably 0.5% to 3%, of a surfactant, with water being present as the remainder or ad 100%.

There may be one or more of the stated ingredients present. Thus, for example, only a single color pigment or else a mixture of different color pigments may be present. Besides one or more color pigments there may also be one or more water-soluble dyes present.

The color pigments may be incorporated into the nail varnish in the form of a pigment preparation, such as a paste-like aqueous dispersion, for instance. It is equally possible, however, to add the pigments in powder form and to treat the resulting nail varnish precursor in a ball mill or comparable apparatus in order to disperse the color pigments.

All of the particles included in the nail varnish, more particularly the color pigments, are capable of entering capillaries, which is to say that they have a size smaller than the internal width of the pores in the application element, which is configured, for example, in the manner of a wick and which is formed, for example, of a sintered polymeric material or of a fibrous material, i.e., a material consisting of a multiplicity of fibers. While this is the case right from the start for the polymeric dispersions used or for the polymeric particles included therein, with particle sizes in the nanometer range, the color pigments and the phyllosilicate added preferably in powder form have particle sizes of less than 20 μm. In the case of the color pigments, for example, there is a particle size distribution of D90<10 μm.

It has surprisingly emerged that the phyllosilicate, whose primary function is that of a dispersant, ensures that the nail varnish has a storage stability of at least 6 weeks, independently of the nature of the color pigments used. In the case of pigment mixtures, therefore, any change in the original mixing ratio of the pigments as a result of differences in sedimentation is prevented. A further surprise was that the phyllosilicate, despite swelling in water, does not, or not significantly, increase the viscosity of the nail varnish, and so the production of nail varnishes having a viscosity below the aforementioned viscosity limit of 50 mPas (Brookfield LV, DV III Ultra Rheometer, spindle: 40, RPM: 10, 25° C.) is readily possible. A final, further surprise was that there are virtually no adverse interactions with the film former such as hindering of film formation.

Phyllosilicates which ensure the aforementioned advantageous properties and effects to a particular degree are lithium magnesium sodium silicates. The phyllosilicates are added usefully in powder form to the initial mixture of the varnish constituents. In order to promote fine division and dispersion of the phyllosilicate, organic polyphosphate may be admixed as an additive, with its fraction—based on the phyllosilicate content—being 2% to 25%, ideally 2% to 10%. The polyphosphate is usefully combined with the phyllosilicate to form a preparation which is added to the nail varnish or to the initial mixture of the varnish constituents.

The polymeric dispersion used in a nail varnish must adhere well to the nail surface and must form a thin, robust, and smooth film. Oftentimes there is a situation where a polymer or a polymeric dispersion prepared from it does adhere well to the nail surface and does form thin and smooth films, but these films do not exhibit any gloss, or any satisfactory gloss. If, however, as is the case with one preferred variant embodiment, the nail varnish contains a surfactant with a fraction of 0.5% to 3%, preferably an anionic surfactant, more particularly an alkoxyalkane, or a polyalkylene glycol ether, for instance, films are produced which have good gloss. The best results in terms of the gloss effect are achieved with a polyoxyethylene-(20) oleyl ether.

Used as film formers in nail varnishes are polymeric dispersions which contain at least one polymer from the group of polyacrylates, polyurethanes, and polyesters or corresponding copolymers and/or hybrid polymers. One problem with film formers for the intended use in question is that, following application, the films produced using them have a tackiness which lasts often for minutes, and for this and other reasons they dry slowly. This situation is different with a nail varnish of the invention wherein the film former contains a polymeric dispersion based on an aliphatic urethane-acrylate hybrid polymer, more particularly a urethane-2-polymethyl methacrylate hybrid polymer. The polymeric dispersions have a solids fraction or polymeric fraction of 20% to 60%. Following application, a nail varnish of this kind exhibits no tackiness and has completely dried after no more than about 10 seconds. A drawback with the stated polymeric dispersions, however, is that they form films having a relatively matt appearance. This can be remedied nevertheless by a particularly preferred combination of the polymeric dispersions in question with a surfactant of the type identified earlier on above; in other words, with a nail varnish modified accordingly, films can be produced that are not tacky, but dry quickly, and that are glossy.

Additives to the nail varnish are, in particular, preservatives such as, for example, phenoxyethanol and caprylyl glycol, fungicides, or care substances.

In accordance with an embodiment of the invention, the surfactant is a nonionic surfactant.

In accordance with another embodiment of the invention, the nail varnish has an aqueous pigment preparation having a solids content of 20% to 60% by weight.

### DESCRIPTION OF THE INVENTION

The table below lists four formulas A to D of the invention, and also a comparative formula V. The corresponding nail varnishes were applied to fingernails of test subjects and were investigated for the uniformity, the initial drying time, the post-application tackiness, and the gloss of the nail varnish films. Moreover, a specified amount of the nail varnishes was stored in a container for 6 weeks at a temperature of 20° C., then applied to a substrate of the aforementioned kind and subjected to color comparison with films produced using unstored—that is, freshly prepared—nail varnishes. The results of the tests can likewise be seen from the table below. In comparison to the nail varnish V, the nail varnishes B to D do not have a tacky consistency and dry substantially more quickly, this being attributable to the film formers present in the form of a dispersion of an aliphatic urethane-acrylate hybrid polymer: in the working examples, a urethane-2-polymethyl methacrylate hybrid polymer dispersion. Intrinsically, the films produced therewith are matt, this also applying in attenuated form to the film according to formula V. The addition of a surfactant (polyoxyethylene-20) oleyl ether) to the nail varnish, however, produces films with a high gloss (A, C, and D).

For the formulas A and D, which contain a phyllosilicate added preferably in powder form, a color change deriving from sedimentation effects is not apparent after storage for 6 weeks.

| | V | A | B | C | D |
|---|---|---|---|---|---|
| Humectant: ethanol | 10.0% | 10.0% | 10.0% | 10.0% | 10.0% |
| Preservative: | 0.5% | 0.5% | 0.5% | 0.5% | 0.5% |
| Dermosoft octiol ¹⁾ | | | | | |
| Preservative: | 0.5% | 0.5% | 0.5% | 0.5% | 0.5% |
| phenoxyethanol | | | | | |
| Water, demineralized | 39.0% | 37.8% | 39.0% | 38.0% | 37.8% |
| Surfactant: BRIJ-O20 | | 1.0% | | 1.0% | 1.0% |
| ss-BR ²⁾ | | | | | |
| Phyllosilicate: Laponite | | 0.2% | | | 0.2% |
| XLS ³⁾ | | | | | |
| Film former: Syntran | 40.0% | 40.0% | | | |
| 5760 PE302-7 ⁴⁾ | | | | | |
| Film former: Hybridur | | | 40.0% | 40.0% | 40.0% |
| 875 ⁵⁾ | | | | | |
| Film former: ammonium | | | | | |
| acrylate copolymer | | | | | |
| Color pigment: | 8.0% | 8.0% | 8.0% | 8.0% | 8.0% |
| Cosmenyl carmin ⁶⁾ | | | | | |
| Cosmenyl black ⁶⁾ | 2.0% | 2.0% | 2.0% | 2.0% | 2.0% |
| | 100.0% | 100.0% | 100.0% | 100.0% | 100.0% |
| Test result | | | | | |
| Uniform film | | − | # | + | ++ |
| Initial drying time | − ~60 s | − ~60 s | + ~10 s | + ~10 s | + ~10 s |
| Tackiness after | − yes | ++ yes | ++ no | ++ no | ++ no |
| application | | | | | |
| Gloss | + | ++ | # matt | ++ | ++ |
| Color change after | − | ++ | − | − | ++ |
| storage | | | | | |

Key to the table: ++ very good/+ good/# acceptable/− poor
Manufacturers:
¹⁾ Dr. Straetmans Chemische Produkte GmbH, D-22143 Hamburg, Germany;
²⁾ Croda GmbH, D-41334 Nettetal Kaldenkirchen, Germany;
³⁾ Rockwood Clay Additives GmbH, D-85368 Moosburg, Germany;
⁴⁾ Interpolymer GmbH, D-67454 Hassloch, Germany
Syntran = styrene/acrylate/ammonium/methacrylate copolymer;
⁵⁾ Air Products and Chemicals Inc., Allentown, USA,
⁶⁾ Clariant, D-65926 Frankfurt, Germany.

A further example of color pigments are those under the trade name Covarine, for instance type W, from Sensient, F-95310 Saint Quen L'Aumone, France.

All percentage figures in the table and elsewhere are percentages by weight.

1. A nail varnish, comprising:
a composition having a viscosity of less than 50 mPas (Brookfield LV, DV III Ultra Rheometer, spindle: 40, RPM: 10, 25° C.), said composition including:
20% to 85% by wt. of an aqueous dispersion of an aliphatic urethane-acrylate hybrid polymer as a film former, with a solids fraction of 20% to 60% by wt.;
5% to 20% by wt. of a humectant;
1% to 10% by wt. of a colorant formed at least partly of a color pigment;
0.1% to 0.5% by wt. of a phyllosilicate;
0.5% to 5% by wt. of a surfactant;
0% to 5% by wt. of an additive; and
remainder is water.

2. The nail varnish according to claim 1, wherein 0.5% to 3% by wt. of said surfactant is present.

3. The nail varnish according to claim 1, wherein 0.5% to 3% by wt. of said additive is present.

4. The nail varnish according to claim 1, wherein said phyllosilicate is a lithium magnesium sodium silicate.

5. The nail varnish according to claim 4, wherein said additive is an organic polyphosphate.

6. The nail varnish according to claim 5, wherein a fraction of said organic polyphosphate is 2% to 10% by wt. of a fraction of said phyllosilicate.

7. The nail varnish according to claim 1, wherein said aqueous dispersion of said aliphatic urethane-acrylate hybrid polymer contains at least one polymer from the group consisting of polyacrylates, polyurethanes, and polyesters.

8. The nail varnish according to claim 7, wherein said aqueous dispersion of said aliphatic urethane-acrylate hybrid polymer is an aqueous dispersion of urethane-2-polymethyl methacrylate hybrid polymer dispersion.

9. The nail varnish according to claim 1, wherein said surfactant is a nonionic surfactant.

10. The nail varnish according to claim 9, wherein said surfactant is an alkoxyalkane.

11. The nail varnish according to claim 10, wherein said surfactant is polyalkylene glycol ether.

12. The nail varnish according to claim 11, wherein said surfactant is polyoxyethylene-(20) oleyl ether.

13. The nail varnish according to claim 1, further comprising an aqueous pigment preparation having a solids content of 20% to 60% by wt.

14. The nail varnish according to claim 1, wherein said colorant contains a water-soluble dye.
